(19) United States
(12) Patent Application Publication
(10) Pub. No.: US 2008/0287555 A1
(43) Pub. Date: Nov. 20, 2008

(54) Novel process and catalyst for carbon dioxide conversion to energy generating products

(75) Inventors: Syed Tajammul Hussain, Islamabad (PK); Mohammad Mazhar, Islamabad (PK); Muhammad Hasib-ur-Rehman, Toba Tek Singh (PK)

Correspondence Address: SARFARAZ K. NIAZI, 20 RIVERSIDE DRIVE, DEERFIELD, IL, 60015, US

(73) Assignee: Quaid-e-Azam University, Islamabad (PK)

(21) Appl. No.: 11/751,026

(22) Filed: May 20, 2007

(51) Int. Cl. C07C 27/06

(52) U.S. Cl. 518/712; 518/715

(57) ABSTRACT

A catalytic process and a nano material for the conversion of moist carbon dioxide into methanol, propyne and oxygen have been developed. In the process invented, hydrogen is produced from water in a catalytic reaction, when the moist carbon dioxide enters into the catalytic reactor, resulting in C—O and H—OH bond breakage at a relatively low temperature and at atmospheric pressure in a single step using a combination of catalytic materials comprising at least three metals dispersed on a catalyst support, preferably anatase form of titanium dioxide, to induce a multifunctional surface chemical reaction for the production of oxygenated products such as hydrocarbons of different chain lengths.

### TECHNICAL FIELD

This invention relates to a process for conversion of carbon dioxide using a supported synthetic catalyst mainly composed of a mixed oxide of ruthenium, cobalt and manganese, into methanol, propyne and oxygen as main products, and an apparatus thereof.

### BACKGROUND

The global warming is considered an urgent issue and one of the most important tasks for humanity to deal, with as it could endanger the existence of humans. Carbon dioxide, a large quantity of which has been accumulated in the world due to the use of fossil fuels, accounts for much of the global warming phenomenon. Further, the rate of carbon dioxide emission has recently increased exponentially, and numerous intensive studies have been conducted all over the world to develop techniques for dealing with this issue.

Global warming is the increase in the average temperature of the Earth's near-surface air and oceans in recent decades and its projected continuation. Global average air temperature near the Earth's surface rose 0.74±0.18° C. (1.3±0.32° F.) during the past century (http://www.ipcc.ch/). The Intergovernmental Panel on Climate Change (IPCC) concludes, “most of the observed increase in globally averaged temperatures since the mid-20th century is very likely due to the observed increase in anthropogenic greenhouse gas concentrations,” which leads to warming of the surface and lower atmosphere by increasing the greenhouse effect. Other phenomena such as solar variation and volcanoes have probably had a warming effect from pre-industrial times to 1950, but a cooling effect since 1950. These conclusions have been endorsed by at least 30 scientific societies and academies of science, including all of the national academies of science of the major industrialized countries. The American Association of Petroleum Geologists is the only scientific society that rejects these conclusions, and a few individual scientists also disagree with parts of them.

Climate models referenced by the IPCC predict that global surface temperatures are likely to increase by 1.1 to 6.4° C. (2.0 to 11.5° F.) between 1990 and 2100. The range of values reflects the use of differing scenarios of future greenhouse gas emissions as well as uncertainties regarding climate sensitivity. Although most studies focus on the period up to 2100, warming and sea level rise are expected to continue for more than a millennium even if no further greenhouse gases are released after this date. This reflects the long average atmospheric lifetime of carbon dioxide.

An increase in global temperatures can in turn cause other changes, including sea level rise, and changes in the amount and pattern of precipitation. There may also be increases in the frequency and intensity of extreme weather events, though it is difficult to connect specific events to global warming. Other consequences may include changes in agricultural yields, glacier retreat, reduced summer stream flows, species extinctions and increases in the ranges of disease vectors.

Remaining scientific uncertainties include the exact degree of climate change expected in the future, especially how changes will vary from region to region around the globe. There is ongoing political and public debate regarding what, if any, action should be taken to reduce or reverse future warming or to adapt to its expected consequences. Most national governments have signed and ratified the Kyoto Protocol aimed at combating greenhouse gas emissions.

The greenhouse effect was discovered by Joseph Fourier in 1824 and was first investigated quantitatively by Svante Arrhenius in 1896. It is the process by which absorption and emission of infrared radiation by atmospheric gases warms a planet's atmosphere and surface.

Greenhouse gases create a natural greenhouse effect without which mean temperatures on Earth would be an estimated 33° C. (59° F.) lower, so that Earth would be uninhabitable. It is therefore not correct to say that there is a debate between those who “believe in” and “oppose” the greenhouse effect as such. Rather, the debate concerns the net effect of the addition of greenhouse gases while allowing for associated positive and negative feedback mechanisms.

On Earth, the major natural greenhouse gases are water vapor, which causes about 36-70% of the greenhouse effect (not including clouds); carbon dioxide, which causes 9-26%; methane, which causes 4-9%; and ozone, which causes 3-7%. The atmospheric concentrations of carbon dioxide and methane have increased by 31% and 149% respectively above pre-industrial levels since 1750. This is considerably higher than at any time during the last 650,000 years, the period for which reliable data has been extracted from ice cores. From less direct geological evidence it is believed that carbon dioxide values this high were last attained 20 million years ago. “About three-quarters of the anthropogenic [man-made] emissions of carbon dioxide to the atmosphere during the past 20 years are due to fossil fuel burning. The rest of the anthropogenic emissions are predominantly due to land-use change, especially deforestation.”

The present atmospheric concentration of carbon dioxide is about 383 parts per million (ppm) by volume. Future carbon dioxide levels are expected to rise due to ongoing burning of fossil fuels and land-use change. The rate of rise will depend on uncertain economic, sociological, technological, natural developments, but may be ultimately limited by the availability of fossil fuels. The IPCC Special Report on Emissions Scenarios gives a wide range of future carbon dioxide scenarios, ranging from 541 to 970 ppm by the year 2100. Fossil fuel reserves are sufficient to reach this level and continue emissions past 2100, if coal, tar sands or methane clathrates are extensively used.

Positive feedback effects such as the expected release of methane from the melting of permafrost peat bogs in Siberia (possibly up to 70,000 million tones) may lead to significant additional sources of greenhouse gas emissions not included in IPCC's climate models.

On the other hand environmental problems due to emissions of pollutants from combustion of solids, liquid and gaseous fuels in various stationary and mobile energy systems as well as the emissions from manufacturing plants have also become major global problems involving not only the production of green house gases such as carbon dioxide and methane by also pollutants such as NOₓ, SOₓ and particulate matter. One way to reduce the green house gases would be to convert them to useful clean energy source and thereby controlling the two major sources of pollution and in the process creating a cheaper source of fuel. There are several other motivations listed below for producing chemicals from carbon dioxide.
1. Carbon dioxide is an inexpensive, nontoxic feedstock that can frequently replace toxic chemicals such as phosgene of isocyanates.
2. Carbon dioxide is a renewable feedstock compared to oil or coal.
3. The production of chemicals from carbon dioxide can lead to new industrial productivity.
4. New routes to existing chemical intermediates and products could be more efficient and economical than current methods.
5. The production of chemicals from carbon dioxide could have a small but significant impact on the global carbon balance.

Conversion and utilization of carbon dioxide are important subjects in the field of chemistry for the production of ultra-clean transportation fuels and hydrogen (C₁ chemistry). Several technologies have been proposed for improving the efficiency of energy conversion and utilization of carbon dioxide. Examples of conventional techniques for hydrogenating carbon dioxide in order to fix carbon dioxide include photo-electrochemical techniques utilizing natural energy, such as sunlight, and biochemical techniques utilizing microorganisms. Disadvantageously, the efficiency of these techniques is low, and the rate of processing is much lower than that of carbon dioxide emission. All the workable technologies to date utilize the processes which are expensive and require working at very high temperatures. The most common of the reaction used is the Sabatier Reaction and Water Gas Shift Reaction. The Sabatier reaction or Sabatier process involves the reaction of hydrogen with carbon dioxide at elevated temperatures and pressures in the presence of a nickel catalyst to produce methane and water. Optionally ruthenium on alumina makes a more efficient catalyst. It is described by the following reaction:

CO₂+4H₂→CH₄+2H₂O Equation 1

It was discovered by the French chemist Paul Sabatier. It has been proposed as a key step in reducing the cost of manned exploration of Mars (Mars Direct) through In-Situ Resource Utilization. After producing water by combining hydrogen transported from Earth and carbon dioxide taken from the atmosphere of Mars, oxygen would be extracted from the water by electrolysis and used as a rocket propellant. The stoichiometric propulsion fuel mix ratio is 1:8 hydrogen:oxygen by weight (each pound of hydrogen requires 8 pound of oxygen to burn), and if only the light hydrogen has to be transported, and the heavy oxygen extracted locally, that would result in a very considerable weight savings which would have to be transported to Mars.

Typically, the energy necessary to split water has been electrical energy (electrolysis). In the past decades, in the field of catalytic chemistry, most efforts have been concentrated on the utilization of carbon dioxide as a source of carbon. Only recently it has been proposed that carbon dioxide might also be utilized as an oxygen source or oxidant, because it can be considered to be a nontraditional (mild) oxidant and oxygen transfer agent. It is well known that aqueous carbon dioxide can be electrocatalytically reduced to produce formic acid, and methanol. Early reports of the electrochemical reduction of carbon dioxide date back to 1870's, although the process only gained recent interests when Halmon demonstrated that aqueous carbon dioxide could be reduced on semiconductor surfaces such as p-type gallium phosphide to produce formic acid, formaldehyde and methanol in a photo-assisted electrolytic reaction (Halmon, M; Nature, 275, 115, 1978). The process, however, produced very low current densities and required high overvolatages.

Hori et al., have shown that carbon dioxide can be electrochemically reduced on the variety of metals and observed the following activity: indium>tin>zinc>lead>copper>gold (Hori, Y; Kamide, N; Suzuki, S; J. Faculty Eng. Chiba, UNiv; 32, 37 (1981).

Kapusta et al., disclose the conversion of carbon dioxide on tin and indium metals in a potassium chloride-sodium bicarbonate solution (Kapusta, S; Hackeman, N; J. Electrochem Soc; 130, 607 (1983). Efficiency of 90% formic acid formation is reported.

Itkulova et al reported the Carbon dioxide reforming of methane over Co—Pd/alumina supported catalyst using a moderate pressure and temperature from 200-1200° C., various oxygenated products (CH₃OH, CO, and hydrogen), (Itkulova, Sh. S; Zhunusova, K. Z; Zakumbaeva, G. D; Bull. Korean Chem. Soc. 26, 12, (2005). This process generates hydrogen by disintegration of CH₄.

Matsuo et al disclose the conversion of carbon dioxide to methane using Hydrosilanes catalyzed by Zirconium-Borane compound in which the hydrogen is supplied from 4Si—H group at 800° C. (Matsuo, T; Kawaguchi, H, JACS, 128, 12362, (2006)

Tan et al disclose a composite catalyst Fe—Zn—Zr/zeolite, which converts carbon dioxide into Isobutane and branched hydrocarbons with hydrogen introduction (Tan, Y; Fujiwara, M; Ando, H; Xu, Q; Souma, Y; Ind. Eng. Chem. Res, 38, 3225, (1999)

Past efforts to produce value added chemicals from carbon dioxide have focused largely on the use of hydrogen (commercially available processes like Sabatier Reaction and Water Gas Shift Reaction). Thus making the process and material is expensive. Thus, there remains a need for a new process and catalytic materials which can generate hydrogen from water by breaking the hydrogen-oxygen bond and utilizing the in situ hydrogen for the production of oxygenates products and hydrocarbons in a single step at atmospheric pressure and at lower temperature.

A large volume of prior art exists in attempts to create an efficient system for converting carbon dioxide into useful products. Given below are some relevant patents issued in this art.

The U.S. Pat. No. 5,904,880: One step conversion of methanol to hydrogen and carbon dioxide, Hsiang-ning Sun: The present invention relates to a one-step method for catalytically reforming methanol with water to produce hydrogen and carbon dioxide using catalysts which do not contain copper oxide and/or chromium oxide, which produce only negligible amounts of carbon monoxide, and which are not rapidly deactivated.

The U.S. Pat. No. 6,248,795: Process of preparing a mixture of dimethyl ether and methanol from carbon dioxide, Jun and Lee. This invention relates to the process of preparing from carbon dioxide a mixture of dimethyl ether and methanol which are useful as clean fuel or raw materials in the chemical industry. More particularly, this invention relates to the process of preparing dimethyl ether and methanol in high yield without by-products such as hydrocarbons by means of chemical conversion of carbon dioxide, which is a major pollutant of the global environment, in the presence of a mixture of catalysts comprising Cu/ZnO-based catalyst and Y-type zeolite catalyst having a strong acidity with the pK.sub.a value of −6.0-−3.0.

The U.S. Pat. No. 5,767,165: Method for converting natural gas and carbon dioxide to methanol and reducing CO₂ emissions, Meyer Steinberg et al. A process for the production of methanol from natural gas containing methane comprising the thermal decomposition of methane and the subsequent reaction of the resulting hydrogen gas with carbon dioxide in a catalyst containing methanol synthesis reactor to produce methanol. Alternative methods include the gasification with carbon dioxide of at least a portion of the carbon produced by the decomposing step, to produce carbon monoxide, which is then reacted with hydrogen gas to produce methanol; or the reforming of a portion of the natural gas feedstock used in the decomposing step with carbon dioxide to produce carbon monoxide and hydrogen gas, which carbon monoxide and hydrogen are then combined with additional hydrogen from the natural gas decomposing step in a methanol synthesis reactor to produce methanol. The methods taught reduce the overall amount of carbon dioxide resulting from the methanol production process.

The U.S. Pat. No. 7,091,251: Process and apparatus for the production of methanol, Alain Guillard et al. In a process for the use of a hydrocarbon feedstock by reacting the feedstock in a reactor with oxygen to form a synthesis gas containing at least carbon monoxide, carbon dioxide and hydrogen and subjecting the synthesis gas to a conversion process comprising an exothermic reaction to produce methanol as a final product in a converter, the converter operating at an operating pressure, the oxygen being provided to the reactor at an oxygen pressure, the synthesis gas is produced at a pressure such that it undergoes at most one compression step with a compression ratio ranging from 1 to 1.7 before entering the converter.

The U.S. Pat. No. 6,376,562: Hybrid catalyst for hydrocarbon synthesis via hydrogenation of carbon dioxide, Son-Ki Ihm et al. The present invention provides a hybrid catalyst which is prepared by mixing a methanol synthesis catalyst with SAPO-type zeolite as a methanol conversion catalyst, and a process for the preparation of hydrocarbons from carbon dioxide by using the hybrid catalyst. The hybrid catalyst of the invention can be used for preparing hydrocarbons having a carbon number of more than 2

The U.S. Pat. No. 3,501,516: Method and production of Methanol, Parrish et al.: Hydrocarbons are reformed in the presence of a reforming catalyst with steam as the sole added oxidant; to produce methanol, synthesis gas comprising hydrogen, carbon monoxide, carbon dioxide, water and from 0 to 12% inerts is introduced at high pressure and temperature.

The U.S. Pat. No. 7,064,150: Method for hydrogenating carbon dioxide, treating apparatus, and basic material for hydrogenation, Masayoshi Matsui: A process for hydrogenating carbon dioxide to generate methanol. In the process, a strip of copper base plate is transported by the groups of rotating drive rollers to deposit porous metallic zinc on the copper base plate. Hydrogen is generated from the porous metallic zinc upon electrochemical reactions in the inner space sealed with the above groups of rollers. Simultaneously, zinc oxide and copper oxide catalysts are formed on the porous metallic zinc. Carbon dioxide is introduced into the sealed inner space under high-temperature and high-pressure to generate methanol by hydrogenation.

The U.S. Pat. No. 6,881,758: Process and apparatus for the production of methanol, Alain Guillard et al. In a process-for the use of a hydrocarbon feedstock by reacting the feedstock in a reactor with oxygen to form a synthesis gas containing at least carbon monoxide, carbon dioxide and hydrogen and subjecting the synthesis gas to a conversion process comprising an exothermic reaction to produce methanol as a final product in a converter, the converter operating at an operating pressure, said oxygen being provided to the reactor at an oxygen pressure, the synthesis gas is produced at a pressure higher than the operating pressure of the converter.

The U.S. Pat. No. 4,579,995: Process for the conversion of methanol to hydrocarbons, Charles H. Mauldin: A process combination wherein (1) in a first stage wet methanol, or methanol and water, are reacted over a copper-containing methanol synthesis catalyst at conditions sufficient to convert at least a portion of the feed to essentially hydrogen and carbon dioxide and, (2) in a second stage, the product stream from said first stage and methanol are reacted over a cobalt or ruthenium catalyst, or cobalt-containing or ruthenium-containing catalyst, to produce, at reaction conditions, an admixture of C.sub.10.sup.+linear paraffin and olefins, which can be further refined and upgraded to high quality middle distillate fuels, and other valuable products such as mogas, diesel fuel, jet fuel, lubes, and specialty solvents, especially premium middle distillate fuels of carbon number ranging from about C.sub.10 to C.sub.20.

The U.S. Pat. No. 6,664,207: Catalyst for converting carbon dioxide to oxygenates and processes thereof and therewith, Jianhua Yao et al. A catalyst and process for converting carbon dioxide into oxygenates. The catalyst comprises copper, zinc, aluminum, gallium, and a solid acid.

The U.S. Pat. No. 5,952,540: Process for preparing hydrocarbons, Kyu Wan Lee et al: This invention relates to a process for preparing hydrocarbons, in particular hydrogenation of carbon dioxide over Fe—K/Al₂O₃ catalyst, which is reduced in hydrogen and activated in the mixture of carbon dioxide and hydrogen.

The U.S. Pat. No. 5,070,016: Integrated process for producing ethanol, methanol and butyl ethers, David E. Hallberg: A methanol synthesis and an ethanol synthesis are integrated into a single continuous process with the by-product carbon dioxide generated in the ethanol synthesis being utilized in the methanol synthesis. The methanol synthesis and ethanol synthesis can be further integrated with isobutylene synthesis with by-product hydrogen formed during isobutylene synthesis being used as a raw material in the methanol synthesis. In the preferred embodiments the ethanol synthesis utilizes Zymomonas mobilis bacteria in anaerobic fermentation in order to maximize the amount of carbon dioxide produced in a form which can be utilized in the methanol synthesis, to reduce carbon dioxide emissions and to provide an ethanol product which is highly suitable for reaction with the isobutylene to form ethyl tertiary butyl ether.

### SUMMARY OF INVENTION

In one aspect, the present invention provides a method to produce hydrogen from water by breaking the hydrogen-oxygen bond at relatively low temperature and at atmospheric pressure in a single step.

In another aspect, the present invention includes a heterogeneous catalytic material which breaks hydrogen-oxygen bond of water during the process.

In another aspect, the present invention includes a heterogeneous catalytic material which breaks the carbon-oxygen bond of carbon dioxide and hydrogen-oxygen bond of water simultaneously during the process.

In another aspect, the present invention includes a supported trimetallic catalytic materials which breaks carbon-oxygen bond at low temperature and at atmospheric pressure. At the surface of this catalytic material the hydrogen, oxygen and carbon react to form oxygenated products and hydrocarbons. The oxygenated hydrocarbon can be methanol, or high molecular weight oxygenated products depending on the concentration of the catalyst materials and temperature of reaction, the hydrocarbon gas can be propyne and other hydrocarbons. The carbon dioxide can be obtained as an industrial byproduct, thus providing a mean to recycle the carbon dioxide that would otherwise be released as an atmospheric pollutant, to form useful chemicals.

In another aspect, the present invention includes a simple process for industries for the conversion of carbon dioxide to useful chemicals. The process includes the flow of carbon dioxide through a container filled with water and this moist gas is injected onto the catalyst surface where a surface reaction takes place at 400° C. or lower and at atmospheric pressure for conversion of carbon dioxide to value added chemicals.

In yet another aspect, the present invention includes a catalytic reactor tube or any other form of tube in which the catalytic material is loaded. The value added chemicals thus produced at the outlet of the reactor can be separated and used.

In yet another aspect, the present invention includes catalytic materials which have specific particle size and shape and enough surface area and multimetallic surface sites with well known surface geometry to have a surface chemical reaction to produce substantially pure methanol and propyne and other value added chemicals at 400° C. or lower and at atmospheric pressure.

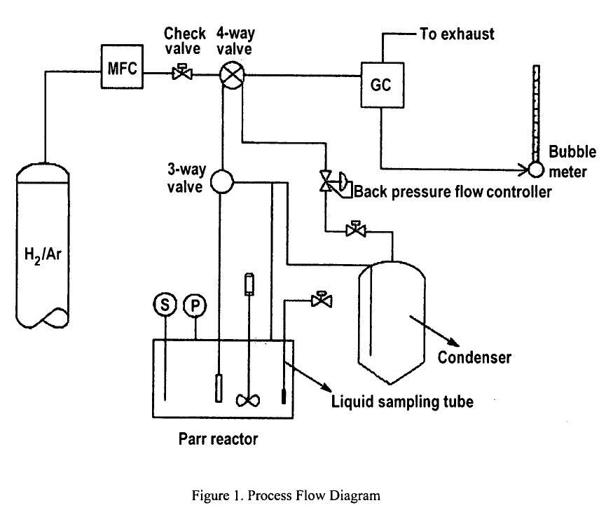

In yet another aspect, the present invention includes catalytic materials which have three metals dispersed on the catalyst support to perform multifunction surface chemical reaction for the production of oxygenated products and hydrocarbons of different chain lengths.

[Insert FIG. 1]

Detailed description of FIG. 1: The process flow for the invention.

FIG. 1 shows a schematic representation of the catalytic reactor system according to the invention.

### DETAILED DESCRIPTION

The present invention discloses a new material composition to produce methanol and related hydrocarbons directly from carbon dioxide. The composition material comprises a mixture of ruthenium 1%, cobalt 5%, manganese 10%, and titanium dioxide 84%

(Composition 1). The catalyst bed was made using ruthenium chloride, manganese chloride, cobalt chloride and dispersing them on to titanium dioxide catalyst support by the process well known in the art.

Whereas optimal results are obtained using the above composition, the selection of reduced metals as described above can be extended to include Pd, Pt, In, Zn, Cu, Ni, W, Fe and Mo. In all instances the reduced metals can be used as metals, as salts or combinations or derivates thereof.

The catalyst support described above could be any ceramic support which may comprise of silica, titania, alumina, zeolite, magnesium oxide and combinations thereof.

The present invention discloses a new method to produce methanol and related hydrocarbons from carbon dioxide. This is accomplished by the process flow diagram described in FIG. 1. Thus, in the method of the present invention, argon and helium are used to flush out the catalytic bed as a common practice in the field, then carbon dioxide is passed through the water and that wet carbon dioxide is supplied to the fixed bed catalytic reactor of 12 inches length, 0.25 inches width operated at 400° C. temperature and at atmospheric pressure as shown in Equation 2, where a surface reaction takes place with the material of the present invention forming methanol, or any other oxygenated products, it should be understood that related hydrocarbon products are also simultaneously produced according to present method.

Carbon dioxide+Water→Methyl Alcohol+Propyne+Oxygen Equation 2

The production of the specific products depends on the percentage composition of the catalytic material. Thus the percentage of production of methanol and propyne or other oxygenated and hydrocarbons products can be readily determined by one skilled in the art.

Advantageously, the product are produces as soon as the surface chemical reaction starts and their production increases with time, reach to maximum after one hour and remains stable during the course of the reaction. The catalyst material of the present invention can be used again and again without any further treatment.

Table 1 lists the production of methanol and propyne with time at 100 mL/min moist carbon dioxide flow at the catalytic reactor bed as calculated from on line GC/MS analysis using the catalyst, Composition-1.

TABLE 1
Representing the percentage selectivity of the products

| | Percentage of | |
|---|---|---|
| | Methanol | Percentage of |
| Time (Hrs) | Production | Propyne Production |
| 0.5 | 10 | 17.5 |
| 1.0 | 15 | 23 |
| 1.5 | 17 | 28 |
| 2.0 | 19 | 30 |
| 3.0 | 19 | 30 |

The methanol and propyne, whose identity were confirmed through mass spectrometry and FTIR analysis, produced from the process allows ready preparation of a whole array of their use as alternate energy resource as fuel and additional source of burning gas at the door step of the carbon dioxide producing industries.

In a preferred embodiment, carbon dioxide is recovered as an industrial by product. By “industrial by product” is meant from industrial sources, such as power plant and other hydrocarbon consuming sources, calcinations of lime stone (cement industries), fermentation processes and other industrial processes which generate carbon dioxide. The recovered carbon dioxide is provided through a flow controller system and transferred through water reservoir and controller on to the catalytic bed for its conversion to methanol, propyne and oxygen. The unreacted carbon dioxide is separated from the reaction products and recycled continuously. The invention described and claimed herein is not to be limited in scope by the specific embodiments herein disclosed. Any equal embodiment is intended to be within the scope of this invention would apply as well. Indeed, various modification of the invention in addition to those shown and described herein will become apparent to those skilled in the art from the foregoing description of the present invention. Such modification are also intended to fall within the scope of the appended claim.

All references cited in the present application are incorporated by reference in their entirety.

What we claim is:

1. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method of converting carbon dioxide into straight chain, C1-C4, hydrocarbons by contacting a mixture of catalytic materials comprising of ruthenium, cobalt and manganese or their salts thereof dispersed onto a catalyst support system with wet carbon dioxide gas.

18. The method of claim 1 wherein said catalytic materials additionally include Pt, Pd, Co, Ag, Au, In, Ce, W, Mo, or Fe or their salts and combinations thereof.

19. The method of claim 1, wherein said catalytic materials are doped by potassium, sodium, cerium and combinations thereof.

20. The method of claim 1 wherein the concentration of said catalytic materials ranges between 1 and 25% of each weight by weight.

21. The method of claim 1 wherein said catalytic materials are in the form of powder, discs or pallets.

22. The method of claim 1 wherein said catalytic materials have the particle size ranging between 10 and 100 nm.

23. The method of claim 1 wherein said catalytic materials are heated to between 50 and 1000° C., more preferably between 400-800° C., prior to contacting them with wet carbon dioxide.

24. The method of claim 1 wherein said catalyst support system comprises of anatase form of titanium dioxide, silica, zeolite, alumina, magnesium oxide, carbon and combinations of thereof.

25. The method of claim 1 wherein said catalyst support system has a surface area ranging between 10-1200 m²g⁻¹.

26. The method of claim 1 wherein said catalyst support system has the particle size ranging between 10 and 200 nm.

27. The method of claim 1 wherein said catalytic materials are in the form of powder, discs or pallets.

28. The method of claim 1 wherein said wet carbon dioxide is contacted with catalytic materials at a flow rate ranging between 60 mL/min to 60,000 mL/min.

29. The method of claim 1 wherein said wet carbon dioxide has the temperature between 80-90° C. prior to contacting with said catalytic materials.

30. The method of claim 1 wherein said straight chain hydrocarbon is methanol.
